(19) United States
(12) Patent Application Publication
(10) Pub. No.: US 2025/0325872 A1
(43) Pub. Date: Oct. 23, 2025

(54) GAIT ANALYSIS METHOD AND APPARATUS USING AUGMENTED REALITY INTERACTIVE SPORTS DEVICE

(71) Applicant: Twohands Interactive Inc., Busan (KR)

(72) Inventor: Wangyun YOO, Busan (KR)

(21) Appl. No.: 18/562,276

(22) PCT Filed: Mar. 11, 2023
(86) PCT No.: PCT/KR2023/003338

§ 371 Date: Dec. 19, 2024

(30) Foreign Application Priority Data
Jan. 12, 2023 (KR) 10-2023-0004697

(51) Int. Cl. A63B 24/00; A63B 71/06

(52) U.S. Cl. CPC A63B 24/0006; A63B 24/0021; A63B 71/0622; A63B 2024/0025; A63B 2024/0037; A63B 2071/0638; A63B 2220/10; A63B 2220/22; A63B 2220/62; A63B 2220/805

(57) ABSTRACT

The present disclosure relates to a gait analysis method using an augmented reality interactive sports device and to be more specific, a gait analysis method which is implemented by an augmented reality interactive sports device which projects an image on a floor and tracks a user' motion using a Lidar sensor unit to control an interaction between contents and a user including (1) displaying a gait path on the floor by means of the augmented reality interactive sports device; (2) measuring foot position information of the user who moves along the gait path using the Lidar sensor unit which laser-scans the plane at a predetermined height from the floor; and (3) analyzing a gait straightness and a gait cadence from the user's foot position information measured in the step (2) and a measurement time.

Further, the present disclosure relates to a gait analysis apparatus using an augmented reality interactive sports device and to be more specific, a gait analysis apparatus includes a gait path generating unit which transmits and receives data with the augmented reality interactive sports device which projects an image on a floor and tracks a user' motion using a Lidar sensor unit to control interaction between contents and the user, and displays a gait path on the floor by means of the augmented reality interactive sports device, a gait measuring unit which measures foot position information of the user who moves along the gait path using the Lidar sensor unit which laser-scans the plane at a predetermined height from the floor; and an gait analysis unit which analyzes a gait straightness and a gait cadence from the user's foot position information measured in the gait measuring unit and a measurement time.

According to the gait analysis method and apparatus using an augmented reality interactive sports device proposed by the present disclosure, a gait path is displayed on a floor using an augmented reality interactive sports device, foot position information of a user who moves along the gait path is measured to analyze and provide gait straightness and gait rhythm to perform the gait analysis simply and easily and at a lower cost by utilizing an augmented reality interactive sports device used for various sports, without using an attachable sensor or a controlled laboratory environment to help identify incorrect waling habits and develop correct walking habits.

### BACKGROUND

### Field

The present disclosure relates to a gait analysis method and apparatus, and more particularly, to a gait analysis method and apparatus using an augmented reality interactive sports device.

### Description of the Related Art

Gait analysis is used for clinical decision-making in musculoskeletal nervous system disorders, post-treatment evaluation, evaluation of prostheses and orthotics, and many biomechanical studies and provides a comprehensive evaluation by measuring several aspects of gait and include kinematic analysis, dynamic electromyography, and energy expenditure measurement. Generally, gait pattern extraction devices are often used for rehabilitation training to restore the sense of walking in patients suffering from gait disorders due to central nervous system damage such as stroke or cerebral palsy, or for gait posture correction training for users who need gait posture correction.

According to the gait analysis method of the related art, information is acquired by attaching an attachable sensor (for example, an inertial sensor such as gyro sensor or an acceleration sensor) onto a body so that the attachable sensor needs to be attached for every measurement, which limits the ability to analyze the gain in daily life. Further, various sensors, a controlled laboratory environment, and equipment such as analysis devices are necessary so that the gait analysis is a difficult test due to the high cost.

Recently, as modern people spend longer periods of time sitting, scoliosis, pelvic distortion, and flat feet are increasing, and these problems affect the walking. Specifically, it is important for children or teenagers to detect and correct incorrect posture early and develop correct walking habits. However, it is not easy to access the gait analysis method of the related art due to its cost so that there is a limitation in that it is not possible to lightly test the gait for correct walking habits.

Accordingly, if there is a technique which analyzes the gait more simple, easily, and at a lower cost, it may help identify the incorrect waling habits and develop the correct walking habits.

In the meantime, as a related art of the present disclosure, Korean Registered Patent No. 10-2233395 (entitled Augmented reality interactive sports device using Lidar sensor, registered on Mar. 23, 2021) has been disclosed.

The above-described background arts are technical information acquired by the inventor for the contents to be disclosed or derived from the contents to be disclosed so that it cannot be referred to as known arts disclosed to the general public prior to the filing of the contents to be disclosed.
(Patent Document 1) Korean Registered Patent No. 10-2233395 (Mar. 13, 2021)

### SUMMARY

The present disclosure has been provided to solve the above-described problems of the methods proposed in the related art and an object of the present disclosure is to provide a gait analysis method and apparatus using an augmented reality interactive sports device which display a gait path on a floor using an augmented reality interactive sports device, measure foot position information of a user who moves along the gait path to analyze and provide a gait straightness and a gait cadence to perform the gait analysis simply and easily and at a lower cost by utilizing an augmented reality interactive sports device used for various sports, without using an attachable sensor or a controlled laboratory environment to help identify incorrect waling habits and develop correct walking habits.

However, the technical object to be achieved by the present disclosure is not limited to the technical object as described above, and there may be other technical object. Further, even if not explicitly mentioned, objects or effects which can be understood from the solving means or exemplary embodiments are also included.

In order to achieve the above-described object, according to an aspect of the present disclosure, a gait analysis method using an augmented reality interactive sports device is a gait analysis method which is implemented by an augmented reality interactive sports device which projects an image on a floor and tracks a user' motion using a Lidar sensor unit to control an interaction between contents and a user, including (1) displaying a gait path on the floor by means of the augmented reality interactive sports device; (2) measuring foot position information of the user who moves along the gait path using the Lidar sensor unit which laser-scans the plane at a predetermined height from the floor; and (3) analyzing a gait straightness and a gait cadence from the user's foot position information measured in the step (2) and a measurement time.

Desirably, the gait path displayed in the step (1) is configured to pass through a sensing area which is laser-scanned by the Lidar sensor unit at least two times while the augmented reality interactive sports device projects the image.

Desirably, the step (2) includes (2-1) a primary measuring step of measuring foot position information of the user who which moves along the gait path using the Lidar sensor unit and recording the measured foot position information and the measurement time and (2-2) a second measurement step of measuring the foot position information of the user which moves along the gait path using the Lidar sensor unit and recording the measured foot position information and records the measurement time, after completing the first measurement step to perform a plurality of gait measurements.

More desirably, in the step (2), a status display image which displays a progress of the first measurement step and the second measurement step is displayed. Desirably, the step (3) includes (3-1) analyzing lengths and times of the step and the stride, the gait velocity, and the number of steps per minute from the foot position information of the user measured in the step (2) and the measurement time; (3-2) analyzing a gait straightness using the foot position information of the user measured in the step (2) and creating a graph; and (3-3) analyzing a gait cadence based on the step length and time analyzed in the step (3-1) and creating a graph.

Desirably, after the step (3), the method further includes: (4) analyzing a gait stability index from the gait straightness and the gait cadence analyzed in the step (3) and providing the gait stability index by means of the augmented reality interactive sports device.

In order to achieve the above-described object, according to an aspect of the present disclosure, a gait analysis apparatus using an augmented reality interactive sports device is a gait analyzing apparatus including a gait path generating unit which transmits and receives data with the augmented reality interactive sports device which projects an image on a floor and tracks a user' motion using a Lidar sensor unit to control interaction between contents and the user, and displays a gait path on the floor by means of the augmented reality interactive sports device, a gait measuring unit which measures foot position information of the user who moves along the gait path using the Lidar sensor unit which laser-scans the plane at a predetermined height from the floor; and an gait analysis unit which analyzes a gait straightness and a gait cadence from the user's foot position information measured in the gait measuring unit and a measurement time.

Desirably, the gait analysis apparatus further includes an index analyzing unit which analyzes a gait stability index from the gait straightness and the gait cadence analyzed in the step (3) and providing the gait stability index by means of the augmented reality interactive sports device.

According to the gait analysis method and apparatus using an augmented reality interactive sports device proposed by the present disclosure, a gait path is displayed on a floor using an augmented reality interactive sports device, foot position information of a user who moves along the gait path is measured to analyze and provide a gait straightness and a gait cadence to perform the gait analysis simply and easily and at a lower cost by utilizing an augmented reality interactive sports device used for various sports, without using an attachable sensor or a controlled laboratory environment to help identify incorrect waling habits and develop correct walking habits.

In addition, various and beneficial advantages and effects of the present disclosure are not limited to the above-described contents, and can be more easily understood during the process of explaining specific embodiments of the present disclosure.

### BRIEF DESCRIPTION OF THE DRAWINGS

FIG. 1 is a view illustrating a configuration of a gait analysis apparatus using an augmented reality interactive sports device according to an exemplary embodiment of the present disclosure;

FIG. 2 is a view illustrating an example of an augmented reality interactive sports device used for a gait analysis method and apparatus using an augmented reality interactive sports device according to an exemplary embodiment of the present disclosure;

FIG. 3 is a flowchart of a gait analysis method using an augmented reality interactive sports device according to an exemplary embodiment of the present disclosure;

FIG. 4 is a view illustrating a floor on which a gait path is displayed, in a step S100 of a gait analysis method using an augmented reality interactive sports device according to an exemplary embodiment of the present disclosure;

FIG. 5 is a view illustrating a gait path along which a user moves, in a step S100 of a gait analysis method using an augmented reality interactive sports device according to an exemplary embodiment of the present disclosure;

FIG. 6 is a view illustrating an example of displaying a user's location according to a user's movement in a gait analysis method using an augmented reality interactive sports device according to an exemplary embodiment of the present disclosure;

FIG. 7 is a sub flowchart illustrating a step S200 in a gait analysis method using an augmented reality interactive sports device according to an exemplary embodiment of the present disclosure;

FIG. 8 is a view illustrating an example of a status displaying image during a first measuring step in a gait analysis method using an augmented reality interactive sports device according to an exemplary embodiment of the present disclosure;

FIG. 9 is a view illustrating a measured foot position of the user in a gait analysis method using an augmented reality interactive sports device according to an exemplary embodiment of the present disclosure;

FIG. 10 is a sub flowchart illustrating a step S300 in a gait analysis method using an augmented reality interactive sports device according to an exemplary embodiment of the present disclosure; and

FIG. 11 is a view illustrating a screen on which the analysis result is displayed in a step S400 of a gait analysis method using an augmented reality interactive sports device according to an exemplary embodiment of the present disclosure.

### DETAILED DESCRIPTION OF THE EMBODIMENT

Hereinafter, the present invention will be described more fully hereinafter with reference to the accompanying drawings, in which exemplary embodiments of the invention are shown. However, the present invention can be realized in various different forms, and is not limited to the exemplary embodiments described herein. Accordingly, the drawings and description are to be regarded as illustrative in nature and not restrictive. Like reference numerals designate like elements throughout the specification.

Throughout this specification, when it is described that an element is “coupled” to another element, the element may be “directly coupled” to the other element or “indirectly coupled” to the other element through a third element. In addition, unless explicitly described to the contrary, when a part includes an arbitrary element, it will be understood to imply the inclusion of stated elements but not the exclusion of any other elements. Further, it may be also understood that the presence or addition of one or more other features, numbers, steps, operations, elements, parts, or combinations thereof are not precluded in advance.

The following exemplary embodiments are detailed descriptions for better understanding of the present disclosure and do not limit the scope of the present disclosure. accordingly, the invention in the same scope which performs the same function as the present invention will also fall within the scope of the present disclosure.

Further, each configuration, process, step, or method included in each exemplary embodiment of the present disclosure may be shared within the scope of not being technically contradictory to each other.

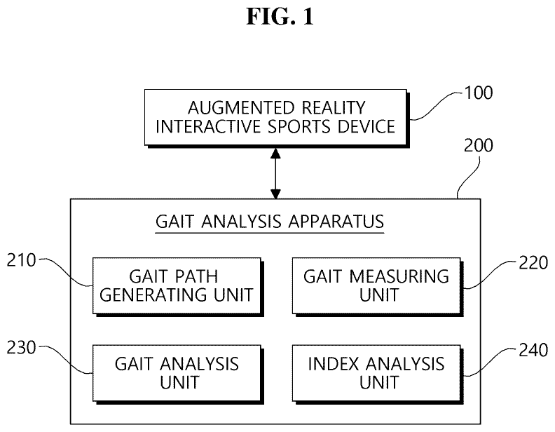

FIG. 1 is a view illustrating a configuration of a gait analysis apparatus 200 using an augmented reality interactive sports device 100 according to an exemplary embodiment of the present disclosure. As illustrated in FIG. 1, a gait analysis apparatus 200 using an augmented reality interactive sports device 100 according to an exemplary embodiment of the present disclosure includes a gait path generating unit 210 which transmits and receives data with the augmented reality interactive sports device 100 which projects an image on a floor and tracks a user' motion using a Lidar sensor unit to control interaction between contents and the user, and displays a gait path on the floor by means of the augmented reality interactive sports device 100, a gait measuring unit 220 which measures foot position information of a user who moves along the waling path using a Lidar sensor which scans a plane with a predetermined height from the floor using laser, and a gait analysis unit 230 which analyzes gait straightness and a gait cadence from the foot position information measured in the gait measuring unit 220 and a measuring time. The gait analysis apparatus 200 further includes an index analysis unit 240 which analyzes a gait stability index from the gait straightness and the gait cadence and provides the gait stability index by means of the augmented reality interactive sports device 100. The gait analysis apparatus 200 using an augmented reality interactive sports device 100 according to an exemplary embodiment of the present disclosure may be embedded in the augmented reality interactive sports device 100 or communicates with the augmented reality interactive sports device 100 in a wired/wireless manner to analyze the gait of the user and provide an analysis result.

The gait straightness refers to the straightness of the user's walking path. It may be an index indicating how straight the walking is, that is, how efficient the path to the goal is.

The gait cadence may refer to the regularity and the consistency of the gait. This represents the rhythm of walking by measuring whether the gait speed is kept constant or the rhythm is constant while walking.

Additionally or alternatively, the gait analysis unit 240 analyzes whether the movement of the user's two feet is symmetrical or asymmetric based on the user's foot position information and measurement time measured by the gait measuring unit 220, and evaluates the balance of the gait to analyze the gait symmetry. The gait symmetry is utilized to identify the necessity of the rehabilitation treatment or identify specific musculoskeletal problems.

Additionally or alternatively, the gait analysis unit 240 infers the change in a ground reaction force through the change in the position of the foot from the user's foot position information measured by the gait measuring unit 220 and the measurement time and also analyzes a pressure applied to the foot while walking.

Additionally or alternatively, the gait analysis unit 240 analyzes a detailed operation pattern while the user is walking, from the user's foot position information measured by the gait measuring unit 220 and the measurement time to acquire useful information for practicing skills of a specific sport or correcting motions required for rehabilitation treatment.

Additionally or alternatively, the gait analysis unit 240 evaluates energy expenditure used during the walking based on the user's foot position information measured by the gait measuring unit 220 and the measurement time. It may help develop a training method to increase exercise efficiency.

The index analysis unit 240 analysis a gait stability index using at least one of the gait straightness, the gait cadence, the gait symmetry, the ground reaction force, or the energy expenditure used during walking. To be more specific, the index analysis unit 240 calculates the gait stability index using an equation comprehensively considering the gait straightness, the gait cadence, the gait symmetry, the ground reaction force, and the energy expenditure used during walking. For example, a linear combination method including a weight as described below may be used.

Gait stability index=w₁(gait straightness score)+w₂(gait cadence score)+w₃(gait symmetry score)+w₄(ground reaction force score)+w₅(energy expenditure score)

Here, w₁, w₂, w₃, w₄, and w₅ are weights indicating importance of the gait straightness, the gait cadence, the gait symmetry, the ground reaction force, and the energy expenditure during walking. The gait stability index may be utilized to provide feedback to the user and improve the rehabilitation treatment or an exercise training program.

The gait straightness score may be a value obtained by measuring how much the gait path of the user matches a predetermined straight path and generally is calculated by quantifying a degree of deviation from the path.

The gait cadence score is a value indicting how consistent a time interval between walking is and is calculated by measuring the regularity and the consistency.

The gait symmetry score is a value indicating how symmetrical the movements of two feet is and is calculates by comparing and analyzing a step length of two feed while walking, a ground contact time, and a foot movement pattern.

The ground reaction force score may be a value indicating a change of force applied to the floor by the foot while walking and is calculated by inferring the change in the ground reaction score through the change in the position of the foot.

The energy expenditure score may be a value indicating an amount of energy consumed while walking and is calculated by measuring or estimating an amount of consumed energy.

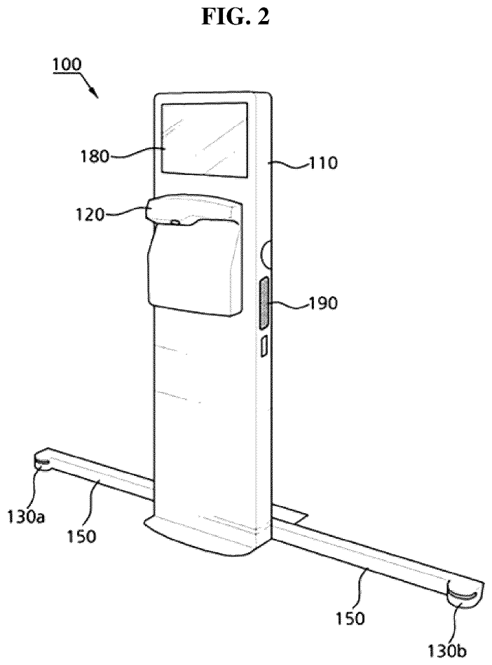

FIG. 2 is a view illustrating an example of an augmented reality interactive sports device 100 used for a gait analysis method and apparatus using an augmented reality interactive sports device 100 according to an exemplary embodiment of the present disclosure. As illustrated in FIG. 2, the augmented reality interactive sports device 100 includes a main body 110, a projector unit 120 which is provided on a front surface of the main body 110 and projects an image for sports contents on the floor, a Lidar sensor unit 130a and 130b installed toward a projecting direction of the projector 120, a motion tracking unit (not illustrated) which tracks a user's motion by recognizing a location of the user according to the sports contents using the Lidar sensor unit 130a and 130b, a sensor connection unit 150 which is connected to a side surface at a lower end of the main body 110 through one end and is connected to the Lidar sensor unit 130a and 130b through the other end, and a controller (not illustrated) outputs the sports contents through the projector 120 to provide sports contents to the user based on the augmented reality and controls the interaction between the sports contents and the user using the user motion tracked by the motion tracking unit 140. The augmented reality interactive sports device 100 may further include a display unit 180 which is provided on the front surface of the main body 110 and receives a selection signal of the sports contents and outputs play information related to the sports contents and a speaker unit 190 which outputs a sound related to the sports contents.

According to the exemplary embodiment of the present disclosure, contents which are output through the projector 120, or controlled by the controller, or output by the display unit may be sports contents including a fitness and exercise program, such as walking, running, aerobics, to analyze and improve the user's gait and a sports program for analyzing a specific motion, such as position movement in ball games, such as soccer and basketball, but are not limited thereto. Therefore, there are a rehabilitation program designed to follow a specific rehabilitation exercise as contents for analyzing and correcting a gait pattern for rehabilitation, a rhythm game or dance program which moves feet to music, an interactive learning program designed to help a user learn about a specific topic, a game or entertainment program which allows a user to interact and enjoy in a virtual environment as an augmented reality game or other forms of entertainment contents, a virtual tour program as contents which allow a user to virtually experience other environment or locations, and a psychotherapy, meditation and breathing exercise program to help a user manage stress and relax.

Here, the Lidar sensor unit 130a and 130b includes two or more Lidar sensors which are connected to both side surfaces of the lower end of the main body 110 through the sensor connection unit 150 to find out the location of each user by avoiding a tracking failure in a laser scanning shadow area due to the related locations of a plurality of users. Further, the motion tracking unit 140 laser-scans a plane at a predetermined height from the ground using the Lidar sensor unit 130a and 130b to recognize the foot position of the user and track the user's motion by tracking the change in the foot position of the user.

Details of the augmented reality interactive sports device 100 may be configured by referring to Korean Registered Patent No. 10-2233395 (entitled Augmented reality interactive sports device 100 using Lidar sensor registered on Mar. 23, 2021) and Korean Registered Patent No. 10-2430084 (entitled Augmented sports system using Lidar sensor registered on Aug. 2, 2022).

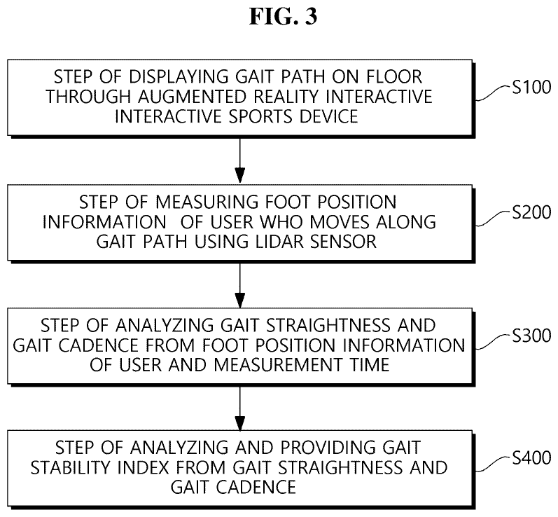

FIG. 3 is a flowchart illustrating a gait analysis method using an augmented reality interactive sports device 100 according to an exemplary embodiment of the present disclosure. As illustrated in FIG. 3, a gait analysis method using an augmented reality interactive sports device 100 according to an exemplary embodiment of the present disclosure is implemented by an augmented reality interactive sports device 100 which projects an image on a floor and tracks a user' motion using a Lidar sensor unit 130a and 130b to control interaction between contents and the user and includes a step S100 of displaying a gait path on the floor by means of the augmented reality interactive sports device 100, a step S200 of measuring foot position information of the user who moves along the gait path using the Lidar sensor unit 130a and 130b, and a step S300 of analyzing a gait straightness and a gait cadence from the user's foot position information and a measurement time. The gait analysis method further includes a step S400 of analyzing and providing a gait stability index from the gait straightness and the gait cadence.

In the step S100, the gait path is displayed on the floor by means of the augmented reality interactive sports device 100. That is, the augmented reality interactive sports device 100 as illustrated in FIG. 2 includes a projector unit 120 which projects a content image on the floor, on the front surface and in the step S100, the gait path is projected and displayed on the floor through the projector unit 120. At this time, the gait path is a path designed for the user to follow for gait analysis and is intuitively displayed with guide lines or arrows.

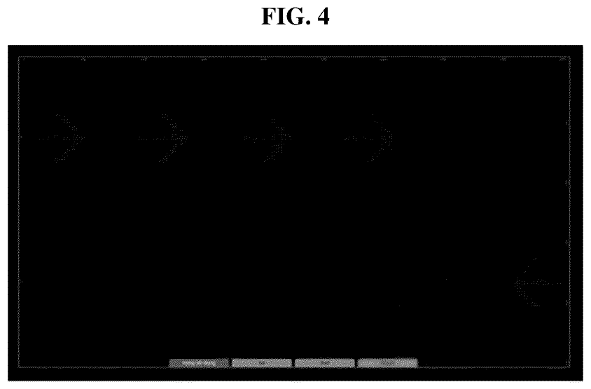

FIG. 4 is a view illustrating a floor on which a gait path is displayed, in a step S100 of a gait analysis method using an augmented reality interactive sports device 100 according to an exemplary embodiment of the present disclosure. As illustrated in FIG. 4, in the step S100 of the gait analysis method using the augmented reality interactive sports device 100 according to the exemplary embodiment of the present disclosure, the gait path is displayed on the floor with a moving arrow. The gait path is displayed with the moving arrows so that when the path is displayed with the guide lines, an intentional walking of a user to move while seeing the guide line is prevented and walking as naturally as possible is induced. Further, the user may intuitively find out the walking direction using the moving arrows.

The gait path displayed in the step S100 is configured to pass through the sensing area which is laser-scanned by the Lidar sensor unit 130a and 130b at least two times while the augmented reality interactive sports device 100 projects the image. To be more specific, the gait path is designed to start from one side of the sensing area, go out of the sensing area, and then re-enter the sensing area again. That is, the gait path is designed to pass through the sensing area two or more times to ensure sufficient gait data.

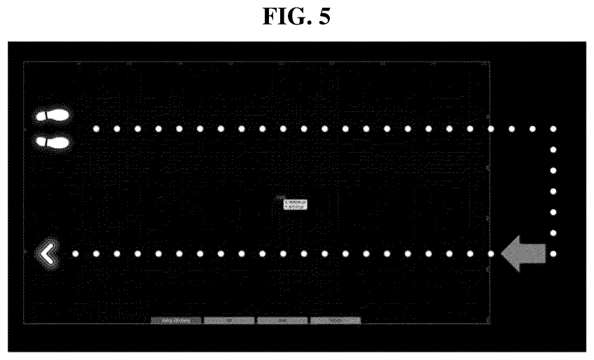

FIG. 5 is a view illustrating a gait path along which a user moves, in a step S100 of a gait analysis method using an augmented reality interactive sports device 100 according to an exemplary embodiment of the present disclosure. As illustrated in FIG. 5, in the step S100 of the gait analysis method using the augmented reality interactive sports device 100 according to an exemplary embodiment of the present disclosure, the gait path is designed to have an inverted ‘E’ shape, starting from a top left side of the sensing area, going out of the sensing area, re-entering the bottom right side, and ending at the bottom left.

The ‘C’ character is one of the letters in Hangul, the Korean alphabet, characterized by its basic linear form. This letter consists of three straight lines: a horizontal line with two short vertical lines attached to its left and right sides. These vertical lines begin at the bottom edge of the horizontal line.

Here, the sensing area is a space set to analyze the gait in a space in which the image is projected by the projector unit 120 and the user position is accurately measured by the Lidar sensor unit 130a and 130b, in the augmented reality interactive sports device 100. In the example illustrated in FIG. 5, a space with 4.5 m wide and 2.5 m tall on the front floor of the augmented reality interactive sports device 100 was set as a sensing area.

In the step S200, foot position information of the user who moves along the gait path is measured using the Lidar sensor unit 130a and 130b which scans the plane at a predetermined height from the floor with laser. To be more specific, the Lidar sensor 130a and 130b of the augmented reality interactive sports device 100 recognizes the user's foot position at a predetermined location from the ground and the motion tracking unit (not illustrated) tracks the user's motion by tracking the change in the user's foot position. That is, the Lidar sensor unit 130a and 130b detects the presence and the position of the user's feet by laser-scanning the plane at a height of approximately 5 cm from the ground and transmits the detected foot position information to the gait analysis apparatus 200. Specifically, as illustrated in FIG. 2, the Lidar sensor unit 130a and 130b is configured by two or more Lidar sensors so that two or more laser scanning areas overlap in the sensing area to accurately recognize positions of a left foot and a right foot of the user.

At this time, in the step S200, the measured foot position information is reflected in real time to display the measured foot position. To be more specific, pixels are set in the sensing area with a predetermined interval and a predetermined recognition image may be displayed in a pixel closest to the user's foot position using the measured foot position information.

The augmented reality interactive sports device 100 may reflect the measured foot position information in real time using various known techniques, such as a Lidar technique, a motion tracking technique, a pixel based position display, an augmented reality technique, 3D image processing, or interactive floor projection.

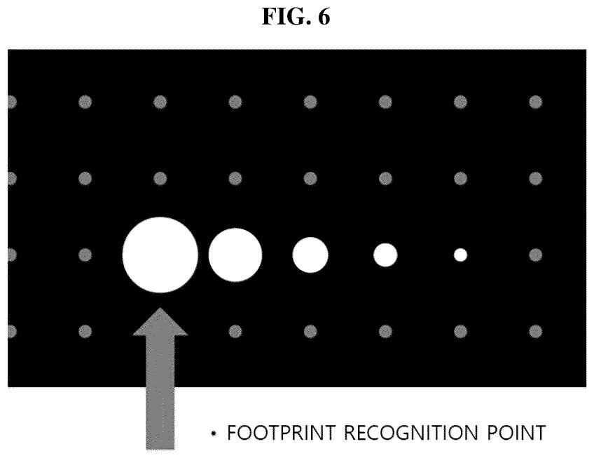

FIG. 6 is a view illustrating an example of displaying a user's location according to a user's movement in a gait analysis method using an augmented reality interactive sports device according to an exemplary embodiment of the present disclosure. As illustrated in FIG. 6, in the gait analysis method using the augmented reality interactive sports device 100 according to the exemplary embodiment of the present disclosure, the sensing area is divided into lattices and recognition images are displayed in the pixels of the lattice point. Here, when the sensing area is divided into lattices, the pixel passes through the gait path and the recognition image is smaller than the lattice size. Further, a recognition image with a white circular shape is displayed in a pixel closest to the user's foot position information measured in step S200. At this time, a predetermined number (five in FIG. 6) of recognition images is displayed in the path direction in which the user has passed from the current position of the user's feet. In this case, the recognition image displayed in the current foot position of the user is displayed to be largest and the size of the recognition image is displayed to be smaller as the distance increases. Such a recognition image gives the user the feeling that the gait measurement is in progress and gives the user the confidence that the location is accurately measured. However, when the foot position of the user is so accurately displayed, it may affect the natural gait so that the recognition image is displayed in a predetermined pixel, rather than the actual foot position of the user.

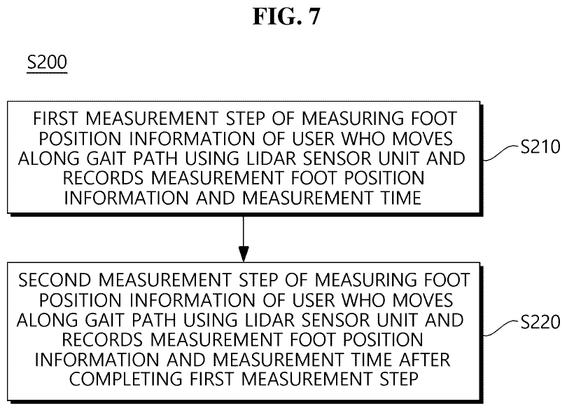

FIG. 7 is a sub flowchart illustrating a step S200 in a gait analysis method using an augmented reality interactive sports device 100 according to an exemplary embodiment of the present disclosure. As illustrated in FIG. 7, the step S200 of the gait analysis method using an augmented reality interactive sports device 100 according to an exemplary embodiment of the present disclosure includes a first measuring step S210 of measuring foot position information of the user who which moves along the gait path using the Lidar sensor unit 130a and 130b and recording the measured foot position information and the measurement time and a second measurement step S220 of measuring the foot position information of the user which moves along the gait path using the Lidar sensor unit 130a and 130b and recording the measured foot position information and records the measurement time, after completing the first measurement step.

The step S210 is a first measuring step of measuring foot position information of the user who which moves along the gait path using the Lidar sensor unit 130a and 130b and recording the measured foot position information and the measurement time.

The step S220 is a second measurement step of measuring the foot position information of the user which moves along the gait path using the Lidar sensor unit 130a and 130b and recording the measured foot position information and records the measurement time, after completing the first measurement step.

That is, in the step S200, accurate gait data is ensured by two measurements of the first measuring step and the second measuring step. Typically, people walk in various ways, such as dragging their feet or walking in small steps so that their footprints overlap or only part of their feet touches the floor. Therefore, valid information must be extracted and analyzed from the measured foot position information. As described above, in order to extract and accurately analyze a valid foot position, sufficient gait data needs to be ensured so that in the step S200, the measurement is performed two or more times.

In step S200, a predetermined area of the foot is set as a threshold value and in the Lidar sensor data, a case in which a predetermined area or more of the foot does not touch the floor, that is, does not reach the threshold value is set to be ignored, so that cases in which only part of the foot touches the floor are filtered out, Further, the threshold value is dynamically adjusted by considering user's walking style, age, gender, height, weight, a type of shoe being worn (for example, boots, pumps, slippers, or sneakers).

Additionally or alternatively, in the step S200, the continuity of foot position is confirmed, and when an abnormally large change in position is detected within a predetermined reference time, this can be regarded as walking in small steps or dragging to be excluded.

Additionally or alternatively, in the step S200, the user's step pattern is learned to distinguish a normal walking pattern from an abnormal pattern (for example, dragging). To this end, an artificial intelligence algorithm may be used.

Additionally or alternatively, in step S200, a 3D model of the foot is generated and only a part which touches the ground flat is considered as valid data.

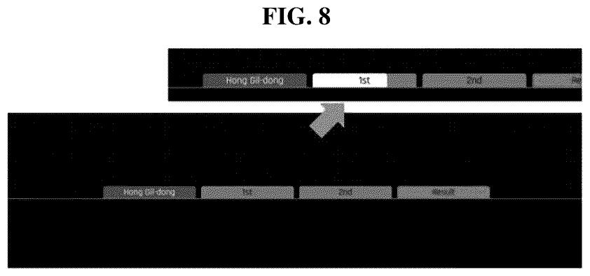

In the meantime, in the step S200, a status display image showing the progress of the first measurement step and the second measurement step may be displayed. FIG. 8 is a view illustrating an example of a status displaying image during a first measuring step in a gait analysis method using an augmented reality interactive sports device 100 according to an exemplary embodiment of the present disclosure. As illustrated in FIGS. 4 and 5, information or a menu for manipulating the augmented reality interactive sports device 100 are displayed on one side of the image which is projected onto the floor. In FIG. 8, the menu displayed at one side of the sensing area is enlarged and a subject to be measured (Hong Gil-dong), a measurement step (1st or 2nd), and a result menu may be included. At this time, the measurement step (1st or 2nd) serves as a status display image which displays the progress of the first measurement step and the second measurement step.

Additionally or alternatively, the status display image includes at least one of a progress displayed by a progress bar or a percentage indicating how much the use has completed the gait path, a measurement step including an icon, number, or color change to visually distinguish the progress of the first measurement and the second measurement, gait data summary including summary information such as measured foot position information and a measurement time, a step length and time, a gait velocity, a number of steps per minute, a gait cadence graph to allow the user to identify the rhythm, including a graph or a chart which visualizes the gait cadence, a gait stability index provided to the user by representing the analyzed gait stability index with number, star rating, or graph, a feedback message including a brief text message or icon to provide a direction for improving the gait of the user, a real-time position including a map or a diagram dynamically indicating a current foot position of the user on the gait path, a warning icon or a message proposed when the user is out of the path or there is a problem in the measurement.

For example, when the user steps on a menu button marked “1st” for more than a predetermined time or kicks the menu button a predetermined number of times or more to start the first measurement step, the button is activated to begin the first measurement step. When the measurement begins, as illustrated at the top of FIG. 8, the “1st” menu button automatically flashes or the color is gradually changed from the left to the right to display the progress of the first measurement step. When the first measurement step ends, a menu button marked “2nd” is automatically activated to start the second measurement step. When the second measurement begins, the “2nd” menu button automatically flashes or the color is gradually changed from the left to the right to display the progress of the second measurement step.

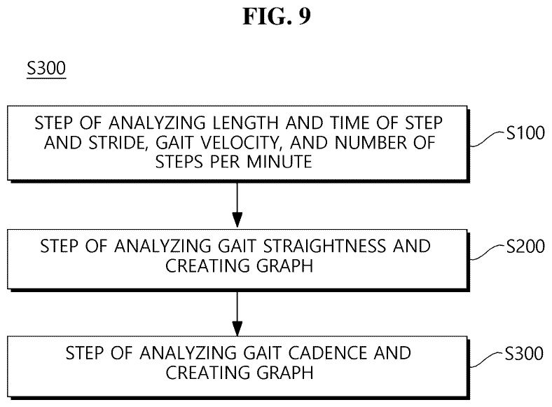

FIG. 9 is a view illustrating a measured foot position of the user in a gait analysis method using an augmented reality interactive sports device 100 according to an exemplary embodiment of the present disclosure. As illustrated in FIG. 9, in the gait analysis method using an augmented reality interactive sports device 100 according to an exemplary embodiment of the present disclosure, whenever the first measurement step and the second measurement step end, the measured foot position of the user is displayed as footprints. That is, since it may affect the user's gait during the measurement, as illustrated in FIG. 6, the foot position of the user is indirectly displayed. However, after completing the measurement, as illustrated in FIG. 9, the foot position is displayed as a sole image in the sensing area of the floor so that the measured foot position is viewed at a glance, thereby increasing the confidence of the user.

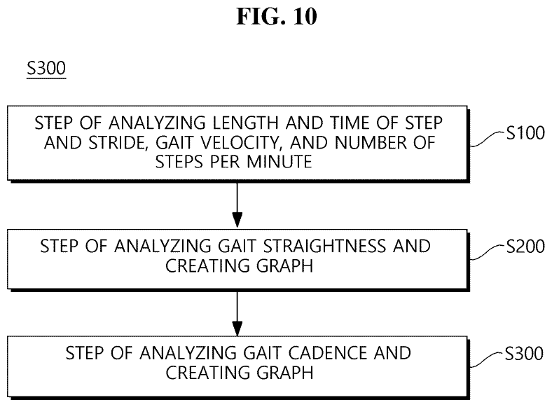

In step S300, the gait straightness and the gait cadence may be analyzed from the foot position information measured in the step S200 and the measurement time. FIG. 10 is a sub flowchart illustrating a step S300 in a gait analysis method using an augmented reality interactive sports device 100 according to an exemplary embodiment of the present disclosure. As illustrated in FIG. 10, the step S300 of the gait analysis method using an augmented reality interactive sports device 100 according to an exemplary embodiment of the present disclosure includes a step S331 of analyzing a length and a time of a step and a stride, a gait velocity, and a number of steps for minute, a step S320 of analyzing the gait straightness and creating a graph, and a step S330 of analyzing a gait cadence and creating a graph.

In the step S400, the gait stability index is analyzed from the gait straightness and the gait cadence analyzed in the step S300 and is provided by means of the augmented reality interactive sports device 100. At this time, in the step s400, not only the gait stability index, but also the analysis result, such as the gait straightness and the gait cadence analyzed in the step S300 are provided. Here, the analysis result including the gait stability index is provided through the projector unit 120 or the display unit 180 of the augmented reality interactive sports device 100.

FIG. 11 is a view illustrating a screen on which the analysis result is displayed in a step S400 of a gait analysis method using an augmented reality interactive sports device 100 according to an exemplary embodiment of the present disclosure. As illustrated in FIG. 11, in the step S400 of a gait analysis method using an augmented reality interactive sports device 100 according to an exemplary embodiment of the present disclosure, lengths and times of the step and stride, a velocity (a gait distance per second), and a gait cadence (a number of steps per minute) are provided. At this time, in the menu as illustrated in FIGS. 4, 5, and 8, when the user steps or kicks the “result” button, the analysis result as illustrated in FIG. 11 is projected through the projector unit 120. In some exemplary embodiment, the length and times of the step and stride, the gait velocity, and the gait cadence are displayed on the display unit 180 in real time to provide a measurement value which changes as the user moves along the gait path.

Hereinafter, each step of the step S300 of a gait analysis method using an augmented reality interactive sports device 100 according to an exemplary embodiment of the present disclosure will be described in more detail with reference to FIGS. 10 and 11.

In the step S310, the lengths and the times of the step and the stride, the gait velocity, and the number of steps per minute may be analyzed from the foot position information of the user measured in the step S200 and the measurement time. Here, the step length is a distance traveled with the left or right foot at once and the stride length is a distance configured by one step with a left foot and one step with a right foot. The gait velocity is defined as a gait distance per second and the number of steps per minutes indicates a gait cadence.

In the step S320, the gait straightness is analyzed and is created on the graph using the foot position information of the user measured in the step S200. How far the left and right feet are from the gait center line can be defined as the step or stride width, and this width is an important measurement value for stable gait. In the step S320, the width of the step or stride is calculated using the foot position information of the user and the positions of the left and the right feet are connected based on the gait center line in the order of measuring times to create a graph. As illustrated in the “straightness” graph in FIG. 11, the created gait straightness graph may be provided to the user and data measured in the first measurement step and the second measurement step are displayed with different colors.

In FIG. 330, the gait cadence is analyzed based on the step length and time analyzed in the step S310 and is represented on the graph. The gait cadence is analyzed based on the step length of the left and right feet and the step time. To be more specific, the gait cadence is defined as the number of steps per minute so that the number of steps per minute of the left foot and the right foot are calculated to analyze the gait cadence and create a graph. A gait cadence graph created as represented in the “cadence” graph in FIG. 11 is provided to the user. Gait cadence is information for confirming the degree of use of both feet and is understood by considering the step length as horizontal movement in the walking and the step time as vertical movement in the walking. For example, when a left foot is uncomfortable, the step length and the time of the left foot are relatively short so that the gait cadence may be biased to one side.

In the step S400, the gait stability index is analyzed from the gait straightness and the gait cadence and provides “Gait stability” illustrated in FIG. 11 to the user. The gait stability index is calculated and provided as a score out of 5 by combining the gait straightness and the gait cadence.

In the meantime, the gait analysis method and apparatus using an augmented reality interactive sports device 100 according to an exemplary embodiment of the present disclosure store and manage measurement data such as measured foot position information of the user and an analysis result for every user. Accordingly, when the user searches or accesses with its own account, the gait information history is confirmed and an improvement direction is established, and an improvement level is identified.

As described above, according to the gait analysis method and apparatus using an augmented reality interactive sports device 100 proposed by the present disclosure, a gait path is displayed on a floor using an augmented reality interactive sports device 100, foot position information of a user who moves along the gait path is measured to analyze and provide gait straightness and gait rhythm to perform the gait analysis simply and easily and at a lower cost by utilizing an augmented reality interactive sports device 100 used for various sports, without using an attachable sensor or a controlled laboratory environment to help identify incorrect waling habits and develop correct walking habits.

In the meantime, an exemplary embodiment of the present disclosure may be implemented as a recording medium including a computer program stored in a medium which is executed by a computer or an instruction which is executable by a computer. The computer readable medium may be an arbitrary available medium which is accessed by a computer and includes all of a volatile and non-volatile medium, a removable and non-removable medium. Further, the computer readable medium may include all of a computer storage medium and a communication medium. The computer storage medium includes an arbitrary method for storing information such as a computer readable command, a data structure, a program module, and other data or a volatile and non-volatile, a removable and non-removable medium which is implemented by an arbitrary method or a technique. A communication medium includes a typical computer readable command, a data structure, a program module, or a data or a modified data signal such as a carrier wave or other transmitting mechanism and also includes an arbitrary information transfer medium.

Although the invention has been described with respect to specific embodiments, some or all of its components or operations may be implemented using a computer system having a general-purpose hardware architecture.

The above-description is illustrative only and it is understood by those skilled in the art that the present invention may be modified to a specific type without changing the technical spirit of an essential feature of the present invention. Thus, it is to be appreciated that embodiments described above are intended to be illustrative in every sense, and not restrictive. For example, each component which is described as a singular form may be divided to be implemented and similarly, components which are described as a divided form may be combined to be implemented.

The scope of the present invention is represented by the claims to be described below rather than the detailed description, and it is to be interpreted that the meaning and scope of the claims and all the changes or modified forms derived from the equivalents thereof come within the scope of the present invention.

What is claimed is:

1. A gait analysis method using an augmented reality interactive sports device 100 which is implemented by an augmented reality interactive sports device 100 which projects an image on a floor and tracks a user' motion using a Lidar sensor unit 130a and 130b to control an interaction between contents and a user, the method comprising:
(1) displaying a gait path on the floor by means of the augmented reality interactive sports device 100;
(2) measuring foot position information of the user who moves along the gait path using the Lidar sensor unit 130a and 130b which laser-scans the plane at a predetermined height from the floor; and
(3) analyzing a gait straightness and a gait cadence from the user's foot position information measured in the step (2) and a measurement time.

2. The gait analysis method using an augmented reality interactive sports device 100 according to claim 1, wherein the gait path displayed in the step (1) is configured to pass through a sensing area which is laser-scanned by the Lidar sensor unit 130a and 130b at least two times while the augmented reality interactive sports device 100 projects the image.

3. The gait analysis method using an augmented reality interactive sports device 100 according to claim 1, wherein the step (2) includes:
(2-1) a primary measuring step of measuring foot position information of the user who which moves along the gait path using the Lidar sensor unit 130a and 130b and recording the measured foot position information and the measurement time and
(2-2) a second measurement step of measuring the foot position information of the user which moves along the gait path using the Lidar sensor unit 130a and 130b and recording the measured foot position information and records the measurement time, after completing the first measurement step to perform a plurality of gait measurements.

4. The gait analysis method using an augmented reality interactive sports device 100 according to claim 3, wherein in the step (2), a status display image which displays a progress of the first measurement step and the second measurement step is displayed.

5. The gait analysis method using an augmented reality interactive sports device 100 according to claim 1, wherein the step (3) includes:
(3-1) analyzing lengths and times of the step and the stride, the gait velocity, and the number of steps per minute from the foot position information of the user measured in the step (2) and the measurement time;
(3-2) analyzing a gait straightness using the foot position information of the user measured in the step (2) and creating a graph; and
(3-3) analyzing a gait cadence based on the step length and time analyzed in the step (3-1) and creating a graph.

6. The gait analysis method using an augmented reality interactive sports device 100 according to claim 1, further comprising:
after the step (3),
(4) analyzing a gait stability index from the gait straightness and the gait cadence analyzed in the step (3) and providing the gait stability index by means of the augmented reality interactive sports device 100.

7. A gait analysis apparatus 200 using an augmented reality interactive sports device 100, comprising:
a gait path generating unit 210 which transmits and receives data with the augmented reality interactive sports device 100 which projects an image on a floor and tracks a user' motion using a Lidar sensor unit to control interaction between contents and the user, and displays a gait path on the floor by means of the augmented reality interactive sports device 100,
a gait measuring unit 220 which measures foot position information of the user who moves along the gait path using the Lidar sensor unit 130a and 130b which laser-scans the plane at a predetermined height from the floor; and
a gait analyzing unit 230 which analyzes gait straightness and a gait cadence from the foot position information measured in the gait measuring unit 220 and a measuring time.

8. The gait analysis apparatus using an augmented reality interactive sports device 100 according to claim 7, further comprising:
an index analysis unit 240 which analyzes a gait stability index from the gait straightness and the gait cadence and provides the gait stability index by means of the augmented reality interactive sports device 100.
